(19) United States
(12) Patent Application Publication
(10) Pub. No.: US 2010/0258125 A1
(43) Pub. Date: Oct. 14, 2010

(54) Respirator and/or Anesthetic Device

(75) Inventors: Harri Friberg, Buchs (CH); Jakob Däscher, Buchs (CH)

Correspondence Address: GIBSON & DERNIER LLP, 900 ROUTE 9 NORTH, SUITE 504, WOODBRIDGE, NJ, 07095, US

(73) Assignee: IMT AG, Buchs (CH)

(21) Appl. No.: 12/740,205

(22) PCT Filed: Nov. 12, 2008
(86) PCT No.: PCT/IB2008/054728

§ 371 Date: May 28, 2010

(30) Foreign Application Priority Data
Nov. 13, 2007 (CH) 01758/07

(51) Int. Cl. A61M 16/00

(52) U.S. Cl. 128/204.23

(57) ABSTRACT

A respirator and/or anesthesia device is provided having internal sensors and at least one respirator connection for the patient as well as an output unit, wherein at least one external sensor can be attached to the patient and its measured values that correlate with the measured values of the internal sensors can be displayed and evaluated.

The invention relates to a respirator and/or anesthesia device having an output unit, i.e., a display, a computer, a control program and internal sensors as well as at least one respirator connection for patients.

Existing respirator devices, which are used home care respiration and in intensive care wards in particular, regulate the pressure and/or gas flow to the patient mainly on the basis of sensors for pressure, flow rate and/or volume, which are secured in the device or on the patient. With these sensors, additional specific patient parameters such as respiratory rate, minute volume, etc., are usually also calculated by a controller, and some of them are then also used as control variables for regulating the pressure and/or gas flow to the patient.

The problem with such respirator devices is that the patient may receive the wrong respiration without the physician or therapist noticing this and/or without drawing the attention of the people caring for the patient. The wrong respiration occurs because simple respiration and/or the response of the lungs to respiration may be different than that required by the patient at this time in the respiration.

To investigate these errors in respiration, it has been proposed that additional patient parameters, e.g., ECG, EEG, mechanical chest and diaphragm expansion, oxygen content in the blood/respiratory air, CO₂ content in the blood and/or respiratory air should be measured. These parameters are available to physicians or therapists when they use additional measurement equipment, so-called patient monitoring equipment. This equipment requires not only the additional expenditure for the machines but also diagnostic skill on the part of the operator personnel to draw the proper conclusions from the values provided. Another decisive advantage is that these additional patient parameters are diverse, and it is difficult for even an experienced physician to properly interpret the different values in their various meanings with regard to the main question of whether or not the correct respiration is administered.

It is not obvious how these values, which are measured and reported separately, are mutually related to the standard measured values such as pressure, flow rate and volume of the respirator device. The first inventive idea therefore goes in the direction that a reliable diagnosis can be made only when a correlated synchronous display of measured values of the respirator together with the additional patient parameters is provided.

Effective and reliable respiration of a patient can thus be achieved according to the invention only by including additional patient parameters, which correlate with one another and are shown in the correct evaluation.

In a hospital, these parameters are measured and displayed in part by means of additional devices, so-called patient monitors, at the patient's bed. However, the measured values from such patient monitors in no way correlate with the measured values shown on the respirator. It is therefore impossible to make diagnoses, or at any rate to do so automatically, of whether the patient is under stress because of an error in setting the respiration or whether he is not breathing in synchronization with the respirator device.

Therefore, in a hospital, changes in a patient's respiration status can be detected only to a limited extent with the devices known so far and can hardly be used directly for control of the respirator device.

The object of the present invention is therefore to create a respirator and/or anesthesia device, which will allow optimal respiration of the patient, also detecting changes in the patient's health status, and will recognize whether it is necessary to vary the respiration and/or whether specialized personnel should be alerted.

According to the invention, this is achieved by providing, i.e., making available, at least one external sensor, the measured values of which correlate with the measured values of the internal sensors, i.e., are in a mutual relationship with one another, can be displayed on the display screen and can be evaluated.

The measured values of the internal and external sensors may thus be correlated according to the invention and displayed simultaneously in synchronization and/or used to control the device.

For optimal monitoring and respiration of a patient, it is advantageous that at least one of the external sensors can be attached to the patient. These are, for example, sensors for measuring the CO₂ content in the blood, ECG electrodes and/or elastic chest bands for measuring the expansion of the chest.

These means are essentially known and have been used thus far to determine the momentary condition of the patient. At any rate, however, they require a diagnostician who is able to (manually) adjust a respirator device on the basis of the given measured values of these known sensors.

The output unit expediently has a device (a so-called ArtifactFinder), which compares the correlated measured values continuously with values stored in tables in a data memory, and in the event of a defined match, automatically displays on the display screen the designation assigned to the corresponding tabulated values. This defined match may be absolutely accurate in value or may be within a predefined tolerance range.

The respiration functions of the device are automatically controlled by the control program as a function of the correlated data of the so-called ArtifactFinder. This achieves the result that changes in the patient's health status are detected immediately and the device can respond rapidly accordingly.

With the traditional method, the momentary patient data are usually detected by sensors and sent to the measurement device over cable connections. On the basis of these data, which are evaluated by a physician, the setting is made manually on the respirator device, usually by the physician.

With the inventive approach, however, monitoring and control around the clock, i.e., in 24-hour operation, are made possible. It is therefore expedient for at least one of the sensors that can be attached to the patient to have a wireless connection to the respirator device. This connection may be established via radio, ultrasound or Bluetooth, for example. The patient may therefore also be relatively mobile with his respirator device.

Apart from the patient's stay in an intensive care ward, he is not normally under constant monitoring by nursing staff. To allow continuous monitoring, it is therefore advantageous to provide a monitoring device, which automatically detects irregularities (artifacts) and reports them continuously and/or stores these data in a retrievable memory. Thus, even when there are special occurrences, the causes leading to them can be recognized and an appropriate response may be taken.

If the values measured exceed or fall below the predefined limits, the monitoring device expediently triggers an alarm function. This alarm function may be visual or acoustic or based on vibration of the device.

If the patient requiring respiration and monitoring is at home or in an area of a hospital remote from the local central office, then the triggering of the alarm function is advantageously relayed automatically and immediately to a central office by telephone, Internet or network connections. The auxiliary personnel can thus intervene within a useful period of time and the required measures can be taken.

According to a special embodiment of the invention, the ArtifactFinder preferably informs the patient about his momentary health status continuously and audiovisually. This has the effect that the patient cannot even experience anxiety and stress feelings. This is very advantageous in particular in view of the fact that with traditional devices, the triggering of the alarm sound itself often leads to extreme stress situations.

The present invention will be explained in greater detail below on the basis of a drawing which illustrates the invention as an example.

This drawing and the list of reference numerals are part of the disclosure.

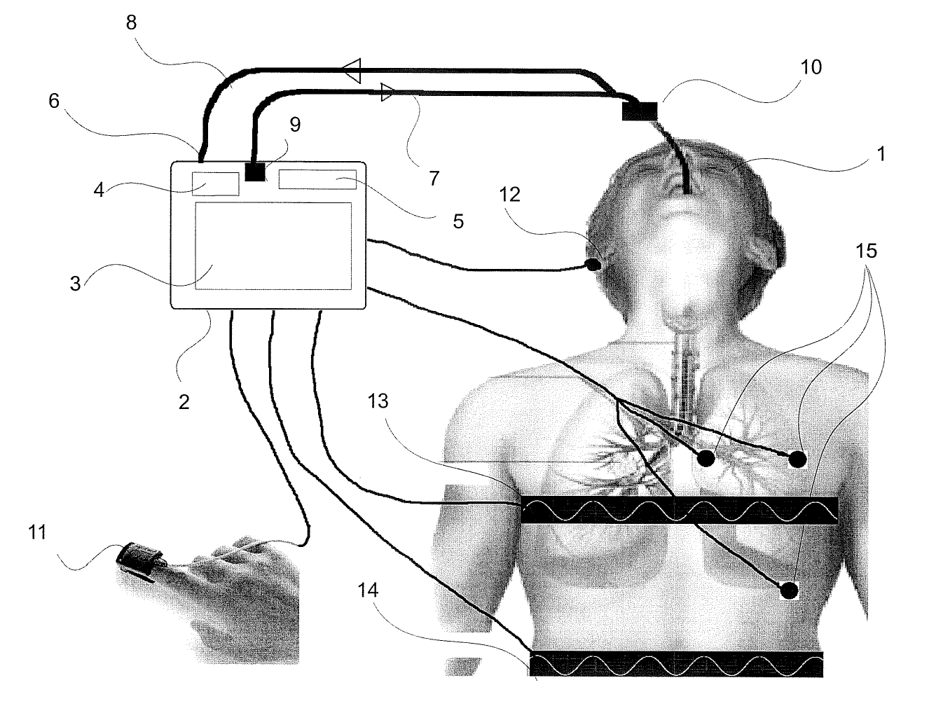

FIG. 1 shows symbolically

a patient and a device for respiration of the patient, including the additional sensors arranged on the patient.

FIG. 1 shows a patient 1 requiring respiration as well as a respirator device 2, which is represented symbolically. The respirator device 2 has a display 3, a computer 4, a control program 5 and at least one respirator connection 6. The respirator connections 6 are connected to flexible tubing for the inhaled air 7 and the exhaled air 8. At the outlet to the tubing for the inhaled air 7, a sensor 9 is provided for measuring the oxygen content of the respiration air. A carbon dioxide sensor 10 continuously measures the CO₂ content of the exhaled air 8.

Various sensors are optionally attached to the patient 1 himself:

A sensor 11 for continuous measurement of the carbon dioxide (CO₂) content in the blood is attached to the finger. Another sensor 12 measures the oxygen (O₂) content of the blood.

An upper chest band 13 (thorax) and a lower chest band 14 are arranged in the area of the chest and in the abdominal area of the patient 1. With the help of these chest bands 13, 14, the respiratory activity of the patient 1 can be monitored.

Additional ECG electrodes are attached in the area of the heart; the cardiac activity of the patient 1 can be monitored with the help of these electrodes.

The measured values of the external sensors 11, 12, 13, 14 and 15 correlate with the measured values of the internal sensors 9, 10, i.e., their mutual relationship is determined and they can be used for control (e.g., adaptive) of the respirator device 2.

### LIST OF REFERENCE NUMERALS

1 patient
2 respirator device
3 display screen
4 computer
5 control program
6 respirator connection
7 inhaled air
8 exhaled air
9 oxygen (O₂) sensor
10 carbon dioxide (CO₂) sensor
11 blood carbon dioxide (CO₂) sensor
12 blood oxygen (O₂) sensor
13 upper chest band (thorax)
14 lower chest band (abdomen)
15 ECG electrodes

1. A respirator and/or anesthesia device having an output unit comprising a display screen, a computer, a control program, internal sensors and at least one respirator connection for a patient, wherein at least one external sensor is provided wherein measured values of the at least one external sensor, correlated with measured values of the internal sensors, are operable to be displayed on the display and evaluated.

2. The device according to claim 1, wherein at least one external sensor is operable to be attached to the patient.

3. The device according to claim 1, wherein the output unit comprises a device which continuously compares the correlated measured values with values stored in a table in a memory, and in the case of a defined match, a designation and/or diagnoses assigned to corresponding tabulated values is/are automatically shown on the display screen.

4. The device according to claim 3, wherein the control program automatically controls respiration functions as a function of the correlated data of the device which continuously compares the correlated measured values with values stored in a table in a memory.

5. The device according to claim 1, wherein at least one external sensor, which can be attached to the patient, comprises a wireless connection to the respirator device.

6. The device according to claim 1, comprising a monitoring device operable to automatically detect irregularities in the measured values and report them continuously and/or stores these data in a retrievable memory.

7. The device according to claim 6, wherein the monitoring device is operable to trigger an alarm function when the measured values exceed or fall below predefined limit values.

8. The device according to claim 7, wherein the control program is connected via a transducer to telephone, Internet or network connections when the alarm function is triggered and includes a program which in the event of an alarm automatically and immediately contacts a central office and forwards the alarm information to the central office.

9. The device according to claim 3, wherein in the respiration state, the device which continuously compares the correlated measured values with values stored in a table in a memory sends the patient signals to inform the patient continuously and audiovisually about the patient's momentary health status.
